SEQUENCE LISTING (2 sequences)

SEQ ID NO: 1 - protein, 449 aa, Homo sapiens
EVQLLESGGGLVQPGGSLRLSCAASGFTFSQYGMDWVRQAPGKGLEWVSGIGPSGGSTVYADSVKGRFTISRDNSKNTLYLQMNSLRAEDTAVYYCTRGGPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQADSFPVTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0290534 A1
(43) Pub. Date: Sep. 23, 2021

(54) METHOD FOR THE PRODUCTION OF FREEZE-DRIED PELLETS COMPRISING AN ANTI-COAGULATION FACTOR XIa (FXIa) ANTIBODY

(71) Applicant: Bayer Aktiengesellschaft, Leverkusen (DE)

(72) Inventors: Stefan Christian SCHNEID, Düsseldorf (DE); Stefan HEKE, Wuppertal (DE); Matthias PLITZKO, Neuenburg (DE)

(73) Assignee: Bayer Aktiengesellschaft, Leverkusen (DE)

(21) Appl. No.: 17/257,827

(22) PCT Filed: Jul. 5, 2019
(86) PCT No.: PCT/EP2019/068071

§ 371 Date: Jan. 4, 2021

(30) Foreign Application Priority Data
Jul. 5, 2018 (EP) PCT/EP2018/068250

(51) Int. Cl. A61K 9/08; A61K 9/19; A61K 9/00; A61K 9/16; A61K 39/395; C07K 16/36; F26B 5/06

(52) U.S. Cl. CPC A61K 9/08; A61K 9/19; A61K 9/0019; A61K 9/1623; F26B 5/065; A61K 9/1652; A61K 39/39591; C07K 16/36; A61K 9/0095; A61K 9/1617

(57) ABSTRACT

A method for the production of freeze-dried pellets comprising an anti-FXIa antibody comprises the steps of: a) freezing droplets of a solution comprising an anti-FXIa antibody to form pellets; b) freeze-drying the pellets; wherein in step a) the droplets are formed by means of droplet formation of the solution comprising an anti-FXIa antibody into a cooling tower which has a temperature-controllable inner wall surface and an interior temperature below the freezing temperature of the solution and wherein in step b) the pellets are freeze-dried in a rotating receptacle which is housed inside a vacuum chamber.

The present invention relates to a method for the production of freeze-dried pellets comprising an anti-coagulation factor XIa (FXIa) antibody, the method comprising the steps of: a) freezing droplets of a solution comprising an anti-FXIa antibody to form pellets; and b) freeze-drying the pellets. The present invention further relates to a method for reducing the reconstitution time of freeze-dried pellets comprising an anti-FXIa antibody and to freeze-dried pellets comprising an anti-FXIa antibody obtainable by the method according to the present invention.

In 1964 Macfarlane and Davie & Ratnoff [Macfarlane R G. An enzyme cascade in the blood clotting mechanism, and its function as a biochemical amplifier. Nature 1964; 202: 498-9; Davie E W, Ratnoff O D. Waterfall sequence for intrinsic blood clotting. Science 1964; 145: 1310-2.] introduced their cascade hypotheses for the process of blood coagulation. Since then, our knowledge of the function of coagulation in vivo has grown. In the last years, the theory of two distinct routes, the so called the extrinsic and intrinsic pathway, that initiate coagulation and converge in a common pathway, ultimately leading to thrombin generation and fibrin deposition, has been revised. In the current model initiation of coagulation occurs when the plasma protease activated factor VII comes into contact and by this forms a complex with Tissue Factor (TF). This Tissue Factor-FVIIa complex can activate the zymogen FX into its active form FXa, which on his part can convert prothrombin (coagulation factor I I) into thrombin (I la). Thrombin, a key player in coagulation, in turn can catalyze the conversion of fibrinogen into fibrin. Additionally, thrombin activates specific receptors expressed by platelets, which leads to the activation of the latter. Activated platelets in combination with fibrin are essential for clot formation and therefore are fundamental players of normal hemostasis.

The second amplification route is formed by the coagulation factor XI (FXI). It is well confirmed that FXI is, like the other members of the coagulation cascade, a plasma serine protease zymogen with a key role in bridging the initiation phase and the amplification phase of blood coagulation in vivo [Davie E W, Fujikawa K, Kisiel W. The coagulation cascade: initiation, maintenance, and regulation. Biochemistry 1991; 30:10363-70; Gailani D, Broze Jr G J. Factor XI activation in a revised model of blood coagulation. Science 1991; 253:909-12; Kravtsov D V, Matafonov A, Tucker El, Sun M F, Walsh P N, Gruber A, et al. Factor XI contributes to thrombin generation in the absence of factor XI I. Blood 2009; 1 14: 452-8.3-5]. The coagulation Factor XI (FXI) is synthesized in the liver and circulates in the plasma as a disulfide bond-linked dimer complexed with High Molecular Weight Kininogen (HMWK). Each polypeptide chain of this dimer is approximately 80 kD. The zymogen Factor XI is converted into its active form, the coagulation factor Xla (FXIa), either via the contact phase of blood coagulation or through Thrombin-mediated activation on the platelet surface. During this activation of factor XI, an internal peptide bond is cleaved in each of the two chains, resulting in the activated factor Xla, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor 11/Thrombin activation.

FXI deficiency usually does not lead to spontaneous bleeding, but is associated with increased risk of bleeding with hemostatic challenges, while the severity of bleeding correlates poorly with the plasma level of FXI. Severe FXI deficiency in humans has certain protective effects from thrombotic diseases. Yet, a high level of FXI has been associated with thrombotic events. Inhibition of FXI has therefore been proposed as a novel approach in the development of new antithrombotics to achieve an improved benefit-risk ratio.

WO 2013/167669 discloses antibodies capable of selectively binding to the activated form of plasma factor XI, FXIa, thereby inhibiting platelet aggregation and associated thrombosis. These antibodies were found not to compromise hemostasis.

Like many other biopharmaceuticals, immunoglobulins are not stable in solution over longer time periods. Freeze-drying, also known as lyophilization, is a process for drying thermo- and/or hydrolysis-sensitive material via sublimation of ice crystals into water vapor, i.e. via the direct transition of water from the solid phase into the gas phase.

In conventional processes, freeze-drying is usually performed in standard freeze-drying chambers comprising one or more trays or shelves within a (vacuum) drying chamber. Vials can be filled with the product to be freeze-dried and arranged on these trays. These dryers typically do not have temperature controlled walls and provide non-homogeneous heat transfer to the vials placed in the dryer chamber. Especially those vials which are positioned at the edges exchange energy more intensively than those positioned in the center of the plates, due to radiant heat transfer and gas conduction in the gap between the wall of the chamber and the stack of plates/shelves. This non-uniformity of energy distribution leads to a variation of freezing and drying kinetics between the vials at the edges and those in the center, and could result in variation in the activities of the active contents of the respective vials and product yield losses. To ensure the uniformity of the final product, it is necessary to conduct extensive development and validation work both at laboratory and production scales.

WO 2006/008006 A1 is concerned with a process for sterile manufacturing, including freeze-drying, storing, assaying and filling of pelletized biopharmaceutical products in final containers such as vials. The described process combines spray-freezing and freeze-drying and comprises the steps of: a) freezing droplets of the product to form pellets, whereby the droplets are formed by passing a solution of the product through frequency assisted nozzles and pellets are formed from said droplets by passing them through a counter-current flow of cryogenic gas; b) freeze-drying the pellets; c) storing and homogenizing the freeze-dried pellets; d) assaying the freeze-dried pellets while they are being stored and homogenized; and e) loading the freeze-dried pellets into said containers.

WO 2013/050156 A1 describes a process line for the production of freeze-dried particles under closed conditions comprising at least a spray chamber for droplet generation and freeze congealing of the liquid droplets to form particles and a bulk freeze-dryer for freeze-drying the particles, the freeze-dryer comprising a rotary drum for receiving the particles. Further, a transfer section is provided for a product transfer from the spray chamber to the freeze-dryer. For the production of the particles under end-to-end closed conditions each of the devices and of the transfer section is separately adapted for operation preserving sterility of the product to be freeze-dried and/or containment.

WO 2013/050161 A1 discloses a process line for the production of freeze-dried particles under closed conditions, the process line comprising a freeze-dryer for the bulk ware production of freeze-dried particles under closed conditions, the freeze-dryer comprising a rotary drum for receiving the frozen particles, and a stationary vacuum chamber housing the rotary drum, wherein for the production of the particles under closed conditions the vacuum chamber is adapted for closed operation during processing of the particles. The drum is in open communication with the vacuum chamber and at least one transfer section is provided for a product transfer between a separate device of the process line and the freeze-dryer, the freeze-dryer and the transfer section being separately adapted for closed operation, wherein the transfer section comprises a temperature-controllable inner wall surface.

Therapeutic antibodies may require administration of high doses in limited volumes and thus high concentrations of the antibody in the final solution to be administered, which results in impracticably long reconstitution times of up to several hours for conventionally freeze-dried products, restricting the applicability of freeze-drying under such circumstances. A method for the production of anti-FXIa antibody comprising freeze-dried pellets having a shortened reconstitution time would be favorable. Furthermore, a freeze-drying method for the production of anti-FXIa antibody comprising freeze-dried pellets that avoids damages to the anti-FXIa antibody during processing and thus binding affinity losses would be desirable. In particular, variations in activity (e.g. binding affinity) among individual pellets should be avoided. Preferably, it should be possible to carry out such a freeze-drying method for the production of anti-FXIa antibody comprising pellets under conditions of strict separation from the outside to ensure sterility—meaning that cooling by a counter or concurrent cooling flow of a cryogenic gas such as liquid nitrogen would need to be avoided. Last, a reproducible process yielding homogenous freeze-dried pellets of a narrow size and weight distribution would offer major advantages for further handling. The present invention has the object of providing such a method. None of the referred to prior art references disclose such a method.

The above stated object is achieved according to the present invention by a method for the production of freeze-dried pellets comprising an anti-FXIa antibody, the method comprising the steps of:

a) freezing droplets of a solution comprising an anti-FXIa antibody to form pellets;
b) freeze-drying the pellets;
wherein in step a) the droplets are formed by means of droplet formation of the solution comprising an anti-FXIa antibody into a cooling tower which has a temperature-controllable inner wall surface and an interior temperature below the freezing temperature of the solution and in step b) the pellets are freeze-dried in a rotating receptacle which is housed inside a vacuum chamber.

The operating principle of the method according to the invention has several distinct advantages. Firstly, it should be noted that in the method according to the invention the sprayed droplets of the anti-FXIa antibody comprising solution do not contact a cryogenic gas in a counter-flow fashion such as described in WO 2006/008006 A1. There is no need for introducing a cryogenic gas into the interior space of the cooling tower and hence all handling and sterilization steps for the cryogenic gas can be omitted. All steps of the method according to the invention can be carried out under sterile conditions and without compromising sterility between the individual steps.

Secondly, the method according to the present invention was experimentally found not to result in significant damages to the anti-FXIa antibody, thus avoiding binding affinity losses in the final product. In fact, anti-FXIa antibody comprising freeze-dried pellets obtained by the method according to the present invention exhibited increased binding affinity towards the FXIa antigen as assessed by indirect ELISA compared to anti-FXIa antibody comprising lyophilisates obtained by conventional freeze-drying or the freeze-drying process according to WO 2006/008006. The avoidance of damages to the anti-FXIa antibody allows precise filling of a desired amount of active anti-FXIa antibody within a narrow specified range. Furthermore, the method according to the present invention allows for more flexibility in filing of the freeze-dried pellets in diverse volumes and application systems as compared to standard lyophilization.

Thirdly, by conducting the freeze-drying step in a rotating receptacle inside the vacuum chamber the spatial position of each individual pellet is evenly distributed over time. This ensures uniform drying conditions and therefore eliminates spatial variations of antibody activity, e.g., binding affinity, as would be the case for freeze-dried vials on a rack.

Last, it was surprisingly found that anti-FXIa antibody comprising pellets produced according to the present invention exhibit a considerably shortened reconstitution time in particular as compared to anti-FXIa antibody comprising lyophilisates obtained by conventional freeze-drying but also as compared to pellets obtained by the process disclosed in WO 2006/008006 A1.

Creation of frozen pellets can be performed according to any known technology. Importantly, however, dropping antibody comprising droplets into liquid nitrogen to therein form pellets is to be avoided.

In view of the subsequent freeze-drying step, the frozen pellets favorably have a narrow particle size distribution. Afterwards the frozen pellets can be transported under sterile and cold conditions to a freeze dryer. The pellets are then distributed across the carrying surfaces inside the drying chamber by the rotation of the receptacle. Sublimation drying is in principle possible in any kind of freeze dryers suited for pellets. Freeze dryers providing space for sublimation vapor flow, controlled wall temperatures and suitable cross sectional areas between drying chamber and condenser are preferred.

Details of the anti-FXIa antibody variants which can be employed in the method according to the invention are described below.

The anti-FXIa antibody to be used in accordance with the present invention is capable of binding to the activated form of plasma factor XI, FXIa. Preferably, the anti-FXIa antibody specifically binds to FXIa. Preferably, the anti-FXIa antibody is capable of inhibiting platelet aggregation and associated thrombosis. Preferably, antibody mediated inhibition of platelet aggregation does not compromise platelet-dependent primary hemostasis. In the context of the present invention the term “without compromising hemostasis” means that the inhibition of coagulation factor XIa does not lead to unwanted and measurable bleeding events.

As used herein, “coagulation factor XIa,” “factor XIa”, or “FXIa” refers to any FXIa from any mammalian species that expresses the zymogen factor XI. For example, FXIa can be human, non-human primate (such as baboon), mouse, dog, cat, cow, horse, pig, rabbit, and any other species expressing the coagulation factor XI involved in the regulation of blood flow, coagulation, and/or thrombosis.

As used herein, an antibody “binds specifically to,” is “specific to/for” or “specifically recognizes” an antigen (here, FXIa) if such antibody is able to discriminate between such antigen and one or more reference antigen(s), since binding specificity is not an absolute, but a relative property. In its most general form (and when no defined reference is mentioned), “specific binding” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard colour development (e.g. secondary antibody with horseradish peroxide and tetramethyl benzidine with hydrogenperoxide). The reaction in certain wells is scored by the optical density, for example, at 450 ran. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like.

However, “specific binding” also may refer to the ability of an antibody to discriminate between the target antigen and one or more closely related antigen(s), e.g., homologs, which are used as reference points. For instance, the antibody may have at least at least 1.5-fold, 2-fold, 5-fold 10-fold, 100-fold, 10³-fold, 10⁴-fold, 10⁵-fold, 10⁶-fold or greater relative affinity for the target antigen as compared to the reference antigen. Additionally, “specific binding” may relate to the ability of an antibody to discriminate between different parts of its target antigen, e.g. different domains or regions of FXIa.

“Affinity” or “binding affinity” KD are often determined by measurement of the equilibrium association constant (ka) and equilibrium dissociation constant (kd) and calculating the quotient of kd to ka (KD=kd/ka). The term “immunospecific” or “specifically binding” preferably means that the antibody binds to the coagulation factor XIa with an affinity KD of lower than or equal to 10⁶M (monovalent affinity). The term “high affinity” means that the KD that the antibody binds to the coagulation factor XIa with an affinity KD of lower than or equal to 10⁷M (monovalent affinity). Such affinities may be readily determined using conventional techniques, such as by equilibrium dialysis; by using the BIAcore 2000 instrument, using general procedures outlined by the manufacturer; by radioimmunoassay using radiolabeled target antigen; or by another method known to the skilled artisan. The affinity data may be analyzed, for example, by the method described in [Kaufman R J, Sharp P A. (1982) Amplification and expression of sequences cotransfected with a modular dihydrofolate reductase complementary dna gene. [J Mol Biol. 159:601-621].

As used herein, the term “antibody” includes immunoglobulin molecules (e.g., any type, including IgG, IgE₁ IgM, IgD, IgA and IgY, and/or any class, including, IgG1, IgG2, IgG3, IgG4, IgAI and Ig A2) isolated from nature or prepared by recombinant means and includes all conventionally known antibodies and functional fragments thereof. The term “antibody” also extends to other protein scaffolds that are able to orient antibody CDR inserts into the same active binding conformation as that found in natural antibodies such that binding of the target antigen observed with these chimeric proteins is maintained relative to the binding activity of the natural antibody from which the CDRs were derived.

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-I, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG.

“Functional fragments” of the invention include Fab, Fab′, F(ab′)₂, and Fv fragments; diabodies; linear antibodies; single-chain antibody molecules (scFv); and multispecific antibodies formed from antibody fragments, disulfide-linked Fvs (sdFv), and fragments comprising a VL or VH domain, which are prepared from intact immunoglobulins or prepared by recombinant means.

Antigen-binding antibody fragments may comprise the variable region(s) alone or in combination with the entirety or a portion of the following: hinge region, CH1, CH2, CH3 and CL domains. Also included in the invention are antigen-binding antibody fragments comprising any combination of variable region(s) with a hinge region, CH1, CH2, CH3 and CL domain.

The antibody and/or antigen-binding antibody fragment may be monospecific (e.g. monoclonal), bispecific, trispecific or of greater multi specificity. Preferably, a monoclonal antibody is used. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the homogeneous culture, uncontaminated by other immunoglobulins with different specificities and characteristics. The modifier “monoclonal” indicates the character of the antibody as being obtained from a substantially homogeneous population of antibodies, and is not to be construed as requiring production of the antibody by any particular method.

The antibody or antigen-binding antibody fragment may for instance be human, humanized, murine (e.g., mouse and rat), donkey, sheep, rabbit, goat, guinea pig, camelid, horse, or chicken. Preferably, a human or humanized anti-FXIa antibody is used.

As used herein, “human” antibodies include antibodies having the amino acid sequence of a human immunoglobulin and include antibodies isolated from human immunoglobulin libraries, from human B cells, or from animals transgenic for one or more human immunoglobulin as well as synthetic human antibodies.

A “humanized antibody” or functional humanized antibody fragment is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin.

Suitable antibodies for the method according to the present invention are for instance disclosed in WO 2013/167669. In particular embodiments, the anti-FXIa antibody comprises at least one CDR amino acid sequence as shown in Table 9 of WO 2013/167669. In particular embodiments, the anti-FXIa antibody comprises at least one of the amino acid sequences for the variable light chain domain and at least one of the amino acid sequences for the variable heavy chain domain as shown in Table 9 of WO 2013/167669. In particular such embodiments, the anti-FXIa antibody comprises i) SEQ ID NO: 19 for the amino acid sequence for the variable light chain domain and SEQ ID NO: 20 for the amino acid sequence for the variable heavy chain domain; or ii) SEQ ID NO SEQ ID NO: 29 for the amino acid sequence for the variable light chain domain and SEQ ID NO: 30 for the amino acid sequence for the variable heavy chain domain; or iii) SEQ ID NO: 27 for the amino acid sequence for the variable light chain domain and SEQ ID NO: 20 for the amino acid sequence for the variable heavy chain domain. In particular embodiments, the anti-FXIa antibody is selected from antibodies 076D-M007-H04, 076D-M007-H04-CDRL3-N110D, and 076D-M028-H17 disclosed in WO 2013/167669. In particular preferred embodiments the anti-FXIa antibody is 076D-M007-H04-CDRL3-N110D, herein represented by SEQ ID NO: 1 for the amino acid sequence for the variable heavy chain domain and SEQ ID NO: 2 for the amino acid sequence for the variable light chain domain.

In particular embodiments, the anti-FXIa antibody is conjugated to a further moiety, in particular a drug.

Embodiments and additional aspects of the present invention will be described below. They can be combined freely unless the context clearly indicates otherwise.

For the present invention any anti-FXIa antibody or functional fragment or variant thereof may be processed without the need for further variation of the process itself. For the realization of the advantageous shortening of the time period required for reconstitution, it is however relevant that an anti-FXIa antibody is processed in the method according to the present invention.

The process preferably avoids potential damage to the anti-FXIa antibody polypeptide and thus losses of activity/affinity in the final product.

In a second aspect, the present invention relates to a method for reducing the reconstitution time of freeze-dried pellets comprising an anti-FXIa antibody as compared to anti-FXIa antibody comprising lyophilisates obtained by conventional freeze-drying, the method comprising the steps of:

a) freezing droplets of a solution comprising an anti-FXIa antibody to form pellets;
b) freeze-drying the pellets;
wherein in step a) the droplets are formed by means of droplet formation of the solution comprising an anti-FXIa antibody into a cooling tower (100) which has a temperature-controllable inner wall surface (110) and an interior temperature below the freezing temperature of the solution and wherein in step b) the pellets are freeze-dried in a rotating receptacle (210) which is housed inside a vacuum chamber (200).

In the context of the present invention, the terms “conventional freeze-drying” and “conventionally freeze-dried” refers to a standard freeze-drying process in vials carried out in a standard freeze-drying chamber comprising one or more trays or shelves within a (vacuum) drying chamber and does not include the process step of spray-freezing. Typically, the product to be freeze-dried is filled into vials which are then placed into the (vacuum) drying chamber.

In the context of the present invention, the term “reducing the reconstitution time of freeze-dried pellets as compared to lyophilisates obtained by conventional freeze-drying” is to be understood as a reduction of the time period required for the complete or near complete dissolution of the freeze-dried pellets obtained by the method according to the present invention upon addition of the reconstitution medium, e.g. sterile water, as compared to lyophilisates obtained by conventional freeze-drying. The reconstitution time is particularly reduced by at least 10%, at least 15%, at least 20%, at least 25%, at least 30%, at least 40%, at least 50%, at least 60%, at least 70%, at least 80%, at least 90% or at least 95%. In the context of the present invention, the term “complete or near complete reconstitution/dissolution of freeze-dried pellets” refers to dissolution of at least 98% of the solids content of the freeze-dried pellets in the reconstitution medium, more particularly of at least 98.5% of the solids content of the freeze-dried pellets, most particularly at least 99%, at least 99.5%, at least 99.75% or at least 99.9% of the solids content of the freeze-dried pellets.

In one embodiment of the method according to the invention the method further comprises the steps c) and d) after step b):

c) storing and homogenizing the freeze-dried pellets
d) loading the freeze-dried pellets into containers.

The storing and homogenization step c) can also be performed in the rotating receptacle within the vacuum chamber used for freeze-drying. In step d) user defined amounts of freeze-dried pellets are filled into the final containers. The storage containers are transferred to an isolated filling line and docked at a sterile docking station. The contents of the containers are transferred inside the isolator to the storage of the filling machine. The method according to the invention which results in no or only minimal damage to the processed anti-FXIa antibody allows for precise filling of the desired antibody amount within narrow specified ranges. The method according to the present invention allows for flexible and individualized filling into containers for final use.

In another embodiment of the method according to the invention in step a) the droplets are formed by means of droplet formation of the solution by passing through frequency-assisted nozzles. Preferably the oscillating frequency is ≥200 Hz to ≤5000 Hz, more particularly ≥400 Hz to ≤4000 Hz or ≥1000 Hz to ≤2000 Hz.

Independent of the nozzle being frequency-assisted, the diameter of the nozzle opening can be in the range of from 100 μm to 500 μm, preferably in the range of from 200 μm to 400 μm, very preferably in the range of from 300 μm to 400 μm. Said nozzle diameters result in droplet sizes in the range from about 200 μm to about 1000 μm, preferably in the range of from about 400 μm to about 900 μm, very preferably in the range of from about 600 μm to 800 μm.

In this context a size of “about” a given value, e.g. the upper or lower limit of a given size range, is to be understood as encompassing all droplet sizes deviating up to ±30% from this given value. For example a resulting droplet size of about 400 μm encompasses droplet sizes varying between 280 μm and 520 μm. Similarly, the size range of from about 100 μm to about 500 μm is to be understood as encompassing droplet sizes from 70 mm to 650 μm.

The droplets formed display a certain droplet size distribution around a median value which should be about the one referenced to above.

In the embodiments of the invention where the nozzle is frequency-assisted the variation around the median value may be smaller. In view of the below described effects passing the droplets through a frequency-assisted nozzle is thus of further advantage to further lower potential negative impact on the final freeze-dried pellets. Also in this context, the term “about” a given value is to be understood as encompassing all values deviating up to ±30% from this given value.

Generally droplets of the sizes given above are of advantage, as it was found that the subsequent steps b) to d) can be performed with good maintenance of anti-FXIa antibody affinity.

Without being bound to that, it is hypothesized that smaller droplets freeze too quickly in the freezing step a) due to the much bigger surface to volume ratio and that the fragile anti-FXIa antibody is thereby partially destroyed. Furthermore, smaller droplets result in smaller pellets which have an increased tendency to become electrostatically charged, the latter impairing subsequent handling of such pellets. For example, the fall of smaller electrostatically charged frozen pellets through the cooling tower tends to be less directed resulting in pellets remaining behind in the tower thereby decreasing the product yield. Bigger droplets do not freeze homogenously. Incomplete freezing of the inner core compartment of the droplets results in the frozen pellets to clump at the bottom of the tower, preventing the formation of a homogenous pellet bulk and thus hindering further processing. Inhomogeneous freezing may further result in partial destruction of the anti-FXIa antibody at the outer shell of the frozen pellet and partial destruction of the anti-FXIa antibody in the inner incompletely frozen core during storage.

In another embodiment of the method according to the invention in step a) the inner surface of the cooling tower has a temperature of not warmer than −120° C., preferably ≥−180° C. to ≤−120° C. Preferably the temperature is ≥−160° C. to ≤−140° C.

The above referred to temperatures of ≥−160° C. to ≤−140° C. are optimized for droplet sizes in the range of about ≥600 μm to about ≤800 μm that are frozen while falling a distance of 2 m to 4 m, particularly about 3 m.

Principally, there is no upper limit regarding the falling distance. The inner surface temperature in the cooling tower and the falling distance are suitably chosen such that droplets of a given size are completely frozen over the chosen falling distance. An inner surface temperature in the cooling tower of below −120° C. allows for complete droplet freezing over feasible falling distances.

In another embodiment of the method according to the invention the inner surface of the cooling tower is cooled by passing a coolant through one or more pipes which are in thermal contact with the inner surface. The coolant may be liquid nitrogen or nitrogen vapor of a desired temperature.

In another embodiment of the method according to the invention the pellet size median of the pellets obtained in step a) is about ≥200 μm to about ≤1500 μm. Preferred is a pellet size median of about ≥500 μm to about ≤900 μm.

Pellets of smaller size than 200 μm are less favorable as in those pellets freezing would be faster which may result in damages of the freeze-dried anti-FXIa antibody and thus loss in binding affinity requiring higher target dosage. Furthermore electrostatic influences of the resulting powder increase dramatically at sizes below 200 μm leading to inferior handling properties of the product of the present process, and yield losses due to entrapment of pellets in water vapor can be expected.

Increase of pellet size to more than 1500 μm may endanger complete freezing of the pellet in the described setup and thus impair the overall quality of a later product.

In another embodiment of the method according to the invention the solution comprising an anti-FXIa antibody in step a) has a content of dissolved solids of ≥5 weight-% to ≤30 weight-%. Preferred is a content of dissolved solids of ≥10 weight-% to ≤20 weight-%.

In another embodiment of the method according to the invention the solution comprising an anti-FXIa antibody in step a) has an antibody concentration of ≥5 mg/ml to ≤300 mg/ml, particularly of ≥50 mg/ml to ≤250 mg/ml, more particularly of ≥100 mg/ml to ≤200 mg/ml.

The required concentration of the anti-FXIa antibody for administration may be relatively high, which commonly causes problems of impractically long reconstitution times of conventionally obtained anti-FXIa antibody comprising lyophilisates. The method according to the present invention was experimentally found to yield freeze-dried anti-FXIa antibody comprising pellets that are significantly faster dissolved in reconstitution medium. This finding was entirely unexpected.

In another embodiment of the method according to the invention the solution comprising an anti-FXIa antibody in step a) has the following composition with respect to 100 ml of the solution, the balance being water for injection:

Anti-FXIa antibody ≥0.5 g to ≤30 g

### Trehalose ≥1 g to ≤25 g

### Histidine ≥50 mg to ≤1.5 g

### Glycine ≥50 mg to ≤1.5 g

### Arginine ≥50 mg to ≤5 g

### Polysorbate 80 ≥5 mg to ≤0.5 g

In another aspect the present invention relates to freeze-dried pellets comprising an anti-FXIa antibody obtainable by the method according to the invention. As detailed above, the freeze-dried anti-FXIa antibody comprising pellets obtained by the method according to the present invention show distinctly different characteristics as compared to lyophilisates obtained by conventional freeze-drying or freeze-dried pellets obtained by a similar, spray-freezing-based method as disclosed in WO 2006/008006. In particular, the anti-FXIa antibody comprising freeze-dried pellets obtained by the method according to the present invention show significantly shorter reconstitution times as compared to equivalent anti-FXIa antibody comprising lyophilisates that were generated by subjecting an identical anti-FXIa antibody comprising starting solution (solution comprising an anti-FXIa antibody in process step a)) to conventional freeze-drying or to the freeze-drying method disclosed in WO 2006/008006. Scanning Electron Microscopy (SEM) further revealed morphological differences between the lyophilisates obtained by the three different freeze-drying methods. The pellets obtained by the method according to the present invention are characterized by a particularly homogeneous surface and low occurrence of microcollapses.

In one embodiment of the freeze-dried pellets according to the invention, the freeze-dried pellets comprising an anti-FXIa antibody exhibit a reduced reconstitution time as compared to anti-FXIa antibody comprising lyophilisates obtained by conventional freeze-drying.

The present invention will be further described with reference to the following figures and examples without wishing to be limited by them.

### FIGURES

FIG. 1 schematically shows an apparatus for the method according to the invention.

FIG. 2 graphically depicts the temperature and pressure profile measured over time during conventional freeze-drying (Method 1) of the antibody solution.

FIG. 3 graphically depicts the temperature and pressure profile measured over time during freezing and drying of the antibody solution according to the method described in WO 2006/008006 (Method 2).

FIG. 4 graphically depicts the temperature profile in the cooling tower measured over time during processing of the antibody solution according to the present invention (Method 3).

FIG. 5 graphically depicts the temperature and pressure profile measured over time during freezing and drying of the antibody solution according to the present invention (Method 3).

FIG. 6 shows Scanning Electron Microscopy (SEM) pictures of a pellet produced according to the present invention (Method 3).

FIG. 7 shows Scanning Electron Microscopy (SEM) pictures of a lyophilisate produced according to conventional freeze-drying (Method 1).

FIG. 8 shows Scanning Electron Microscopy (SEM) pictures of a lyophilisate produced according to the freeze-drying process disclosed in WO 2006/008006 (Method 2).

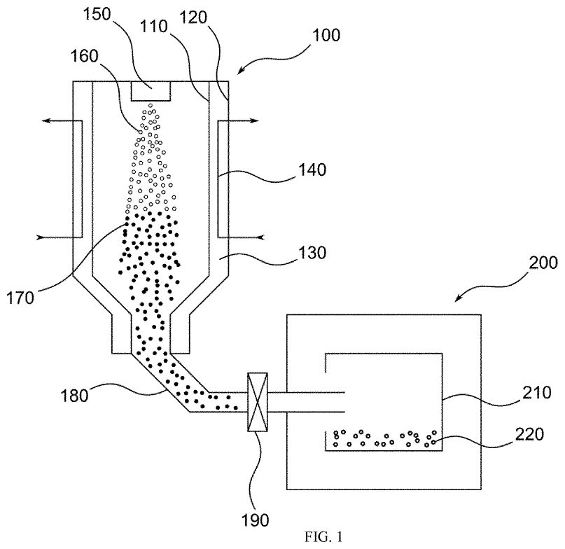

FIG. 1 schematically depicts an apparatus for conducting the method according to the invention. The apparatus comprises, as main components, the cooling tower 100 and the vacuum drying chamber 200.

The cooling tower comprises an inner wall 110 and an outer wall 120, thereby defining a space 130 between the inner wall 110 and the outer wall 120.

This space 130 houses a cooling means 140 in the form of piping. A coolant can enter and leave the cooling means 140 as indicated by the arrows of the drawing.

Coolant flowing through the cooling means 140 leads to a cooling of the inner wall 110 and thus to a cooling of the interior of the cooling tower 100. In the production of frozen pellets (cryopellets), liquid is sprayed into the cooling tower via nozzle 150. Liquid droplets are symbolized in accordance with reference numeral 160.

The liquid droplets eventually solidify (freeze) on their downward path, which is symbolized in accordance with reference numeral 170. Frozen pellets 170 travel down a chute 180 where a valve 190 permits entry into the vacuum drying chamber 200.

While not depicted here, it is of course also possible and even preferred that the chute 180 is temperature-controlled in such a way as to keep the pellets 170 in a frozen state while they are collecting before the closed valve 190.

Inside the vacuum drying chamber 200 a rotatable drum 210 is located to accommodate the frozen pellets to be dried. The rotation occurs around the horizontal axis in order to achieve an efficient energy transfer into the pellets. Heat can be introduced through the drum or via an encapsulated infrared heater. As an end result, freeze-dried pellets symbolized by the reference numeral 220 are obtained.

### EXAMPLES

### Example 1: Lyophilization by Conventional Freeze-Drying

This example describes conventional lyophilization (Method 1) of a liquid high-concentration composition comprising 076D-M007-H04-CDRL3-N110D. The composition comprised a histidine-glycine-arginine buffer system. Trehalose was added as stabilizer. 076D-M007-H04-CDRL3-N110D was formulated at approximately 150 mg/ml in:

20 mM L-Histidine, 50 mM L-Arginine hydrochloride, 50 mM Glycine, 5% trehalose dihydrate, 0.10% polysorbate 80, pH 5.0 (composition 32).

To develop a suitable lyophilization process it was essential to determine the collapse temperature that decided at which temperature the primary drying could be conducted. The collapse temperature was measured using a lyo-microscope (Lyostat 2, Biopharma) by freezing the composition to −50° C. before drawing vacuum (0.1 mbar) and heating the sample with a ramp of 1° C./minute to 20.0° C. While heating up the composition pictures were taken and analyzed until a collapse of the tested system could be observed.

The collapse temperature of 076D-M007-H04-CDRL3-N110D was found to be −14.3° C. and is an essential parameter for selection of the following lyophilization cycle.

The liquid composition 32 comprising anti-FXIa antibody 076D-M007-H04-CDRL3-N110D was processed according to a conventional freeze-drying method (Method 1). The solution containing 150 mg/ml anti-FXIa antibody was filled into 10R type I glass vials and freeze-dried in a conventional vial freeze dryer. A total of 20 vials were filled with 2.25 ml solution per vial, semi-stoppered and loaded into a Virtis Genesis freeze dryer. The solution was frozen to −45° C., and primary drying was performed at +10° C., followed by a secondary drying step at 40° C. The complete freeze drying process required approx. 38 hours. The vials were stoppered within the freeze dryer and sealed directly after unloading.

The details of the lyophilization cycle according to a conventional freeze-drying method (Method 1) for composition 32 are summarized in Table 1.

TABLE 1
Lyophilization cycle of composition 32 (Method 1)

| | Time | Temp | Pressure |
|---|---|---|---|
| | [hh:mm] | [° C.] | [mbar] |
| Loading | 00:01 | 20.0 | 1000 |
| Freezing | 00:30 | −5.0 | 1000 |
| Freezing | 01:00 | −5.0 | 1000 |
| Freezing | 00:40 | −45.0 | 1000 |
| Freezing | 03:30 | −45.0 | 1000 |
| Evacuation | 00:01 | −45.0 | 0.100 |
| Primary drying | 01:00 | 10 | 0.1 |
| Primary drying | 19:00 | 10 | 0.1 |
| Secondary drying | 01:00 | 40 | 0.04 |
| Secondary drying | 10:00 | 40 | 0.04 |

| | | |
|---|---|---|
| Time | Loading | 00:01 |
| Summary | Freezing | 05:41 |
| | Primary drying | 20:00 |
| | Secondary drying | 11:00 |
| | Total | 36:42 |

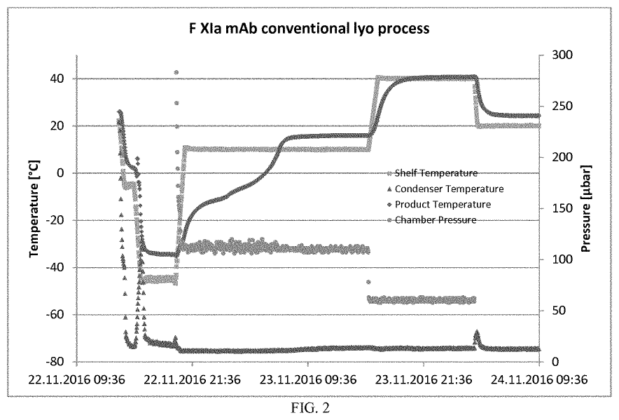

The pressure and temperature profile measured over time during the thus conducted conventional freeze-drying process is graphically depicted in FIG. 2.

The conventional lyophilization method described above resulted in a yellowish cake or powder which can subsequently be reconstituted.

For reconstitution of the lyophilisate 2 ml sterile water for injection as reconstitution medium was injected into each of the vials. The vials were then gently agitated for about 10 to 20 seconds. Reconstitution of this lyophilisate obtained by conventional freeze-drying resulted in a reconstitution time of 137 min.

After reconstitution a clear, yellowish solution without any visible particles was observed. No aggregation or hints of aggregation were detected.

### Example 2: Lyophilization by Two Different Spray-Freeze-Drying Methods

As the reconstitution time of the lyophilisate obtained by a conventional freeze-drying method as described in Example 1 (Method 1) was, with more than 2 hours, unacceptably long, two different other freeze-drying methods were applied and compared to the conventional freeze-drying as described above.

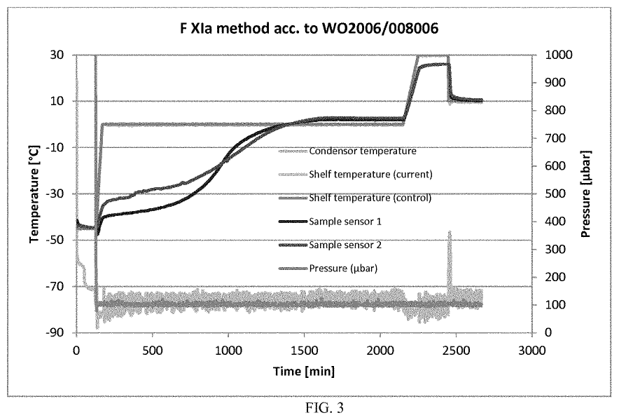

Firstly, the liquid composition 32 comprising anti-FXIa antibody 076D-M007-H04-CDRL3-N110D was processed according to the method described in WO 2006/008006 (Method 2). 138 ml solution containing 150 mg/ml anti-FXIa antibody were sprayed through a 400 μm nozzle and atomized at a frequency of 470 Hz with a rate of about 19.5 g/min and a pressure overlay of 220 mbar. The droplets were frozen in an isolated vessel filled with liquid nitrogen that was positioned approx. 25 cm below the nozzle and stirred throughout the process. After completion of spraying the frozen pellets were removed by pouring the liquid nitrogen through a pre-cooled sieve and placed in a steel rack lined with plastic foil onto the pre-cooled shelves of a Virtis Advantage Pro freeze dryer and lyophilized. Primary drying was conducted at 0° C. shelf temperature over a duration of 33 hours, followed by secondary drying for 5 hours at 30° C. After completion of drying, the dry pellets were instantly transferred into glass bottles which were firmly closed. Subsequently, 520 mg of pellets were weighed into 10R type I glass vials under a dry nitrogen atmosphere. The pressure and temperature profile measured over time during freezing and drying of the antibody solution according to the method described in WO 2006/008006 is graphically depicted in FIG. 3.

Secondly, the liquid composition 32 comprising anti-FXIa antibody 076D-M007-H04-CDRL3-N110D was processed according to the spray-freeze-drying based method for reducing the reconstitution time of freeze-dried pellets according to the present invention (Method 3) which comprises the steps of:

a) freezing droplets of a solution comprising an anti-FXIa antibody to form pellets;
b) freeze-drying the pellets;
wherein in step a) the droplets are formed by means of droplet formation of the solution comprising an anti-FXIa antibody into a cooling tower which has a temperature-controllable inner wall surface and an interior temperature below the freezing temperature of the solution and in step b) the pellets are freeze-dried in a rotating receptacle which is housed inside a vacuum chamber.

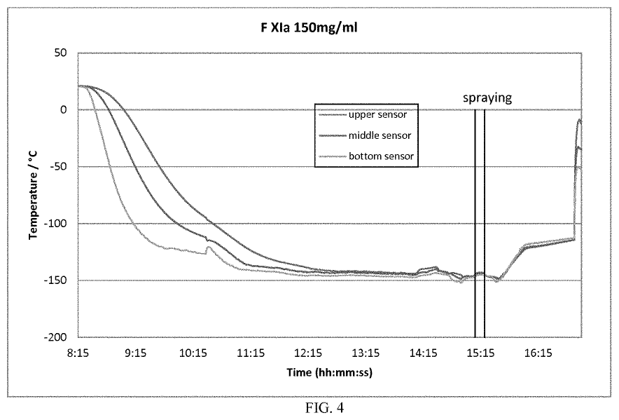

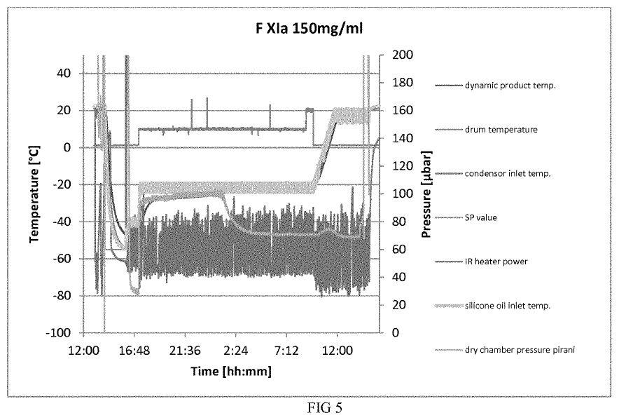

For this purpose, 250 ml solution containing 150 mg/ml anti-FXIa antibody was freeze-dried by spraying the solution into a wall-cooled cooling tower. The spraying nozzle had one aperture with a diameter of 400 μm. This corresponds to a droplet size of about 800 μm. The oscillation frequency was 1445 Hz, the deflection pressure 0.4 bar and the pump was operated at 14 rpm. After completion of drying, the dry pellets were instantly transferred into glass bottles which were firmly closed. Subsequently, 520 mg of pellets were weighed into 10R type I glass vials under a dry nitrogen atmosphere. The temperature profile in the cooling tower measured over time is graphically depicted in FIG. 4. The temperature and pressure profile measured over time during freezing and drying of the antibody solution is graphically depicted in FIG. 5.

The freeze-drying method according to the present invention (Method 3) yielded uniform pellets exhibiting a narrow size and weight distribution and a high surface area. The residual humidity in the pellets obtained by this method was 0.268%. The lyophilisates obtained by conventional freeze-drying (Method 1) comprised 0.15% residual moisture.

Size exclusion chromatography analyses of the pellets obtained by the three different freeze-drying processes are given in the Table 2.

TABLE 2
Size exclusion chromatography analyses of the pellets
obtained by the three different freeze-drying processes

| | SEC | | | |
|---|---|---|---|---|
| | | Sum high | Sum low | |
| | | molecular | molecular | |
| | Dimer | weight (HMW) | weight (LMW) | Monomer |
| Sample | [% area] | aggregates | aggregates | [% Area] |
| Method 3 | 1.66 | 1.82 | 1.20 | 96.96 |
| (as described | | | | |
| herein) | | | | |
| Method 1 | 1.35 | 1.41 | 1.13 | 97.45 |
| (Conventional | | | | |
| Lyophilization) | | | | |
| Method 2 | 1.57 | 1.77 | 1.15 | 97.07 |
| (W02006/ | | | | |
| 008006) | | | | |

Overall, comparable analytical data were obtained by size exclusion chromatography for the three freeze-drying methods.

To determine the quantity of intact antibody relative to the overall proteinaceous components present in the sample, IgG purity was analyzed by Capillary SDS-Gel Electrophoresis (CGE). Test and reference samples were separated by CGE using a bare fused-silica capillary in the presence of sodium dodecyl sulfate (SDS). The test was performed under non-reducing conditions. The separated samples were monitored by absorbance at 220 nm. The intention of the assay was to integrate the peak area of the main peak and analyze the byproducts after reduction.

The results of capillary gel electrophoresis (CGE) and ELISA analyses are given in the Table 3.

TABLE 3
Capillary gel electrophoresis (CGE) and ELISA analyses of the
pellets obtained by the three different freeze-drying processes

| CGE | | | | | |
|---|---|---|---|---|---|
| | IgG | HHL | HH | HL | |
| | [% corr. | [% corr. | [% corr. | [% corr. | |
| Sample | Area] | Area] | Area] | Area] | ELISA |
| Method 3 | 95.82 | 2.51 | 0.38 | 0.18 | 112 (111.95) |
| Method 1 | 95.80 | 2.60 | 0.36 | 0.17 | 101 (100.73) |
| Method 2 | 95.83 | 2.55 | 0.38 | 0.17 | 87 (86.87) |

Reconstitution times of the pellets obtained by the three different freeze-drying methods were compared as follows. 2 ml sterile water for injection as reconstitution medium was injected into each of the vials. After taking photographs the vials were gently agitated for about 10 to 20 seconds. Reconstitution of the pellets over time was visually observed and documented photographically.

The reconstitution times of the pellets obtained by the three different freeze-drying methods are given below:

| Freeze-Drying method | Reconstitution Time | Ab Concentration |
|---|---|---|
| Method 1 | 137 min | 150 mg/ml |
| Method 2 | 16 min | 150 mg/ml |
| Method 3 | 11 min | 150 mg/ml |

The reconstitution of the freeze-dried anti-FXIa antibody comprising pellets obtained with the method according to the present invention (Method 3) was significantly faster than the reconstitution of equivalent anti-FXIa antibody comprising lyophilisates obtained by conventional freeze-drying (Method 1), but also faster compared to freeze-dried pellets obtained according to WO 2006/008006 (Method 2).

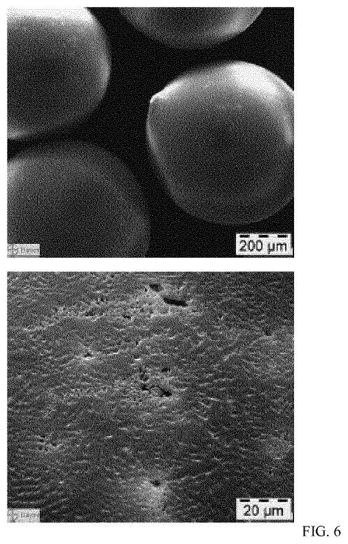

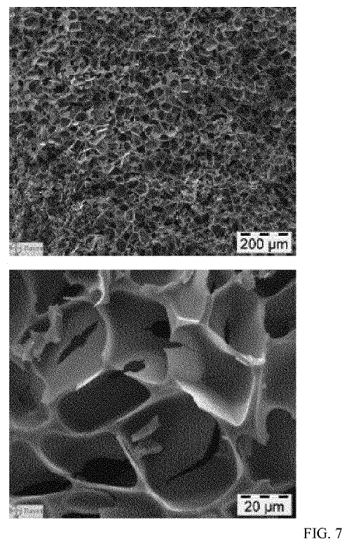

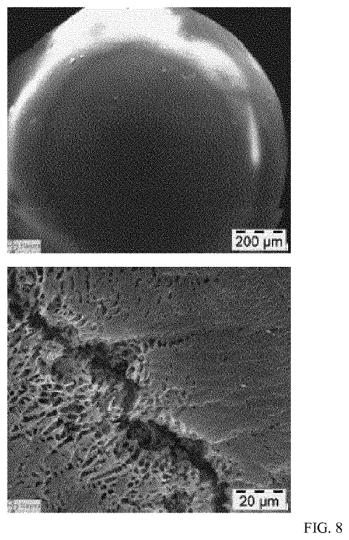

The pellets obtained by the three different freeze-drying methods were thereafter subjected to Scanning Electron Microscopy (SEM) measurements. Therefore, preparation of samples was performed in a glove bag under nitrogen atmosphere, each sample was prepared individually. The sample was placed on a holder and sputtered with gold. Subsequently the scanning electron microscopy measurement was performed. SEM pictures are shown in FIGS. 6 to 8.

It can be seen that the pellets produced pursuant to the method according to the invention display a particularly homogeneous morphology, which may improve handling properties in later process steps.

1. A method for the production of freeze-dried pellets comprising an anti-coagulation factor XIa (FXIa) antibody, the method comprising the steps of:
a) freezing droplets of a solution comprising an anti-FXIa antibody to form pellets; and
b) freeze-drying the pellets;
wherein
in step a) the droplets are formed by means of droplet formation of the solution comprising an anti-FXIa antibody into a cooling tower (100) which has a temperature-controllable inner wall surface (110) and an interior temperature below the freezing temperature of the solution;
and
in step b) the pellets are freeze-dried in a rotating receptacle (210) which is housed inside a vacuum chamber (200).

2. A method for reducing the reconstitution time of freeze-dried pellets comprising an anti-FXIa antibody as compared to anti-FXIa antibody comprising lyophilisates obtained by conventional freeze-drying, the method comprising the steps of:
a) freezing droplets of a solution comprising an anti-FXIa antibody to form pellets; and
b) freeze-drying the pellets;
wherein
in step a) the droplets are formed by means of droplet formation of the solution comprising an anti-FXIa antibody into a cooling tower (100) which has a temperature-controllable inner wall surface (110) and an interior temperature below the freezing temperature of the solution; and
in step b) the pellets are freeze-dried in a rotating receptacle (210) which is housed inside a vacuum chamber (200).

3. The method according to claim 1, further comprising the steps c) and d) after step b):
c) storing and homogenizing the freeze-dried pellets; and
d) loading the freeze-dried pellets into containers.

4. The method according to claim 1, wherein in step a) the droplets are made by means of droplet formation by passing the solution through frequency-assisted nozzles.

5. The method according to claim 4, wherein the oscillating frequency is ≥200 Hz to ≤5000 Hz.

6. The method according to claim 1, wherein in step a) the inner surface (110) of the cooling tower (100) has a temperature of ≤−120° C.

7. The method according to claim 1, wherein the inner surface (110) of the cooling tower (100) is cooled by passing a coolant through one or more pipes (140) which are in thermal contact with the inner surface (110).

8. The method according to claim 1, wherein the pellet size median of the pellets obtained in step a) is about ≥200 μm to ≤1500 μm.

9. The method according to claim 1, wherein the solution comprising an anti-FXIa antibody in step a) has a content of dissolved solids of ≥5 weight-% to ≤30 weight %.

10. The method according to claim 1, wherein the solution comprising an anti-FXIa antibody in step a) has an antibody concentration of ≥5 mg/ml to ≤300 mg/ml.

11. The method according to claim 1, wherein the solution comprising an anti-FXIa antibody in step a) has the following composition with respect to 100 ml of the solution, the balance being water for injection:
Anti-FXIa antibody ≥0.5 g to ≤30 g
Trehalose ≥1 g to ≤25 g
Histidine ≥50 mg to ≤1.5 g
Glycine ≥50 mg to ≤1.5 g
Arginine ≥50 mg to ≤5 g
Polysorbate 80 ≥5 mg to ≤0.5 g

12. Freeze-dried pellets comprising an anti-FXIa antibody obtainable by the method according to claim 1.

13. The freeze-dried pellets comprising an anti-FXIa antibody according to claim 12, wherein the freeze-dried pellets comprising an anti-FXIa antibody exhibit a reduced reconstitution time as compared to anti-FXIa antibody comprising lyophilisates obtained by conventional freeze-drying.
